(19) United States
(12) Patent Application Publication
(10) Pub. No.: US 2019/0282780 A1
(43) Pub. Date: Sep. 19, 2019

(54) SLEEP INDUCING DEVICES AND METHOD THEREOF

(71) Applicant: Young Kwon KWAK, Seoul (KR)

(72) Inventor: Young Kwon KWAK, Seoul (KR)

(21) Appl. No.: 16/354,640

(22) Filed: Mar. 15, 2019

(30) Foreign Application Priority Data
Mar. 16, 2018 (KR) 10-2018-0031125

(51) Int. Cl. A61M 21/02; G06F 3/16

(52) U.S. Cl. CPC A61M 21/02; A61M 2021/0027; G06F 3/165

(57) ABSTRACT

The sleep inducing device includes: an input receiving unit for receiving an input of a user; a background sound output unit for outputting a first signal corresponding to a background music having a melody and a rhythm according to the input of the user; a sleep inducing sound output unit for outputting a second signal having a maximum amplitude at a sleep inducing frequency and corresponding to a sleep inducing sound having a pitch varying to produce a consonance with the melody of the background music; and a mixing unit mixing the first signal and the second signal to output a mixing signal. The user can feel more comfortable while sleeping by changing the pitch of the sleep inducing sound to coincide with the melody of the background music.

### CROSS REFERENCE TO RELATED APPLICATION

The present application claims priority to Korean Patent Application No. 10-2018-0031125, filed Mar. 16, 2018, the entire content of which is incorporated herein by reference.

### BACKGROUND OF THE INVENTION

### 1. Field of the Invention

The present invention relates to a device and a method for inducing sleep. More particularly, the present invention relates to a device and method for inducing sleep using an electroencephalogram tuning.

### 2. Description of Related Art

The brain, which is the central body that controls the mental phenomenon, movement, and senses, exhibits various movements due to the bonding form or activity of the brain nerve cells. Electroencephalogram (EEG) is a spontaneous electric potential in the scalp, which is an electrical signal that measures the electrical changes caused by the activity of brain nerve cells. EEG can be classified into delta waves (0.5-4 Hz), seta waves (4-8 Hz), alpha waves (8-14 Hz), beta waves (14-30 Hz) and gamma waves (30 Hz and higher) depending on the frequency band.

Electroencephalogram (EEG) can be mainly caused by brain waves in a specific frequency band depending on the state of mind and body. For example, in the state of extreme anxiety or excitement of the mind and body, gamma waves appear mainly, and beta waves appear mainly in a tense or stressed state. In the stable state, alpha waves appear mainly. In a drowsy state or a shallow sleep state, seta waves appear mainly, and it is known that delta waves appear mainly in unconscious state in deep sleep.

According to the British scientist Gray Walter, human brain waves are tuned to the same frequency in response to vibrations of a certain frequency. There is a device that emits light and sound at a specific frequency by using the EEG tuning phenomenon and adjusts the state of the brain to a state in which it is good for sleeping or studying. However, users who actually use such a device often complain that the monotonically sound of a specific frequency becomes sleepy even in a situation where the user has to study, or that the sound of a specific frequency becomes interrupts the sleep in a situation where the user has to sleep.

### SUMMARY OF THE INVENTION

The present invention has been made to solve the above problems of the prior art. An object of the present invention is to provide a device, a method and a recording medium which emit a sleeping inducing sound comfortable for a user to hear.

According to a first aspect of the present invention, there is provided a sleep inducing device comprising: an input receiving unit for receiving a user input; a background sound output unit for outputting a first signal corresponding to background music having a melody and a rhythm according to the user input; a sleep inducing sound output unit for outputting a second signal corresponding to a sleep inducing sound whose pitch changes so as to have consonance with the melody of the background music; and a mixing unit for mixing the first signal and the second signal to output a mixing signal.

According to another aspect of the present invention, there is provided the sleep inducing device, further comprising: a memory for storing a plurality of background music data and a plurality of sleep inducing sound data corresponding to the plurality of background music data; wherein the background sound output unit reads background music data corresponding to the user input from the memory and generates the first signal, and the sleep inducing sound output unit reads the sleep inducing sound data corresponding to the background music data from the memory and generate the second signal.

According to another aspect of the present invention, there is provided the sleep inducing device, wherein the sleep inducing frequency is selected within a predetermined frequency range to induce the sleep.

According to another aspect of the present invention, there is provided the sleep inducing device, wherein the sleep inducing frequency is selected between 2 Hz and 10 Hz.

According to another aspect of the present invention, there is provided the sleep inducing device, wherein a frequency per minute corresponding to the sleep inducing frequency is an integral multiple of beats per minute of the background music.

According to another aspect of the present invention, there is provided the sleep inducing device, wherein the sleep inducing frequency is reduced (with a tempo of the background music) after a predetermined time.

According to another aspect of the present invention, there is provided the sleep inducing device, wherein the amplitude of the sleep inducing sound changes in a sinusoidal wave form.

According to another aspect of the present invention, there is provided the sleep inducing device, wherein the pitch of the sleep inducing sound changes when the amplitude of the sleep inducing sound is lower than a preset ratio with respect to the maximum amplitude.

According to another aspect of the present invention, there is provided the sleep inducing device, wherein the mixing unit comprises: a first volume control unit for adjusting a size of the first signal according to a first volume input of the user for adjusting a volume of the background music; and a second volume control unit for adjusting a size of the second signal according to a second volume input of the user for adjusting the volume of the sleep inducing sound independently of the volume of the background music.

According to another aspect of the present invention, there is provided the sleep inducing device, wherein the background music includes an autonomous sensory meridian response (ASMR).

According to another aspect of the present invention, there is provided the sleep inducing device, further comprising: a background natural sound output unit for outputting a third signal corresponding to a background natural sound according to the user input, wherein the mixing unit mixes the first to third signals, and outputs the mixing signal.

According to another aspect of the present invention, there is provided the sleep inducing device, further comprising: a speaker for receiving the mixing signal from the mixing unit and outputting audio mixed with the background music and the sleep inducing sound.

According to another aspect of the present invention, there is provided the sleep inducing device, further comprising: a wireless communication unit for receiving the mixing signal from the mixing unit, converting the mixing signal into a wireless signal, and transmitting the wireless signal to the wireless audio device.

According to another aspect of the present invention, there is provided the sleep inducing device, further comprising: an audio signal output unit for receiving the mixing signal from the mixing unit, converting the mixing signal into an audio signal, and outputting the audio signal.

According to a second aspect of the present invention, there is provided the sleep inducing method comprising: receiving an user input; selecting one of the plurality of background music stored in the memory according to an user input; and mixing a sinusoidal wave sleep inducing sound corresponding to the background music with the background music and outputting them in a sound form; wherein the background music has a melody, the sleep inducing sound has a maximum amplitude at a sleep inducing frequency, and a pitch is changed so as to make a consonance with the melody of the background music.

According to another aspect of the present invention, there is provided the sleep inducing method, further comprising: independently adjusting the volume of the background music and the volume of the sleep inducing sound according to the user input.

According to another aspect of the present invention, there is provided the sleep inducing method, further comprising: selecting one background natural sound of a plurality of background natural sounds stored in the memory according to the user input; and mixing the background music, the sleep inducing sound, and the background natural sound and outputting them in a sound form.

According to a third aspect of the present invention, there is provided the sleep inducing device comprising: a processor; and a memory for storing commands, a plurality of background music data and a plurality of sleep inducing sound data corresponding to each of the plurality of background music data; wherein the processor is operative to perform the following steps: receiving a user input when the commands are executed by the processor; selecting first background music data from a plurality of background music data stored in the memory according to the user input; selecting first sleep inducing sound data corresponding to the first background music data from a plurality of sleep inducing sound data stored in the memory; mixing and outputting a background music having a melody corresponding to the first background music data and a sleep inducing sound of sinusoidal wave corresponding to the first inducing sleep sound data; wherein the sleep inducing sound of sinusoidal wave has a maximum amplitude at a sleep inducing frequency, and the pitch changes so as to make a consonance with the melody of the background music. A fourth aspect of the present invention provides a storage medium storing a computer-readable computer program for executing a sleep inducing method.

According to various embodiments of the present invention, in other to tune the EEG, the sleep inducing sound has a maximum amplitude at a sleep inducing frequency, and the pitch changes so as to make a consonance with the melody of the background music, so that the user who listens to background music along with sleep inducing sound, can comfortably sleep without discomfort. At the moment when the pitch of the sleep inducing sound changes, the amplitude of the sleep inducing sound is synchronized with the minute instant, so that the user may not feel discomfort due to the moment when the pitch of the sleep inducing sound changes.

In addition, by providing an intuitive interface, the user can conveniently perform the sleep inducing method according to the present invention.

The user can conveniently set the most appropriate environment by adjusting the volume of the background music and the volume of the sleep inducing sound independently of each other.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a diagram showing a network configuration of a sleep inducing device according to an embodiment of the invention;

FIG. 2 is a block diagram showing a configuration of a sleep inducing device according to an embodiment of the invention;

FIG. 3 is a diagram for explaining background music and a sleep inducing sound output by the sleep inducing device according to an embodiment of the invention;

FIG. 4 is a flowchart illustrating a sleep inducing method according to an embodiment of the invention;

FIGS. 5A-5E illustrates a user interface screen of a sleep inducing device in which a sleep inducing application program is installed according to an embodiment of the invention;

FIGS. 6A-6B illustrates a user interface screen of a sleep inducing device in which a sleep inducing application program is installed according to an embodiment of the invention; and

FIG. 7 illustrates a user interface screen of a sleep inducing device in which a sleep inducing application program is installed according to an embodiment of the invention.

### DETAILED DESCRIPTION OF THE PREFERRED EMBODIMENTS

Hereinafter, various embodiments will be described in detail with reference to the accompanying drawings so that those skilled in the art can easily carry out the present invention.

However, the technical idea of the present invention can be modified and embodied in various forms, and thus is not limited to the embodiments described in this disclosure.

In the following description of the embodiments of the present invention, a detailed description of related arts will be omitted when it is determined that the technical idea of the present invention may be blurred.

The same or similar components are denoted by the same reference numerals, and redundant description thereof will be omitted.

Throughout the specification, when an element is referred to as being “connected” to another element, it includes not only “directly connected” but also “electrically connected”. Also, in the following description of the embodiments of the present invention, when it is determined that a detailed description of related arts will be made to obscure the technical idea of the present invention, a detailed description of the known art will be omitted.

Some embodiments may be described with functional block arrangements and various processing steps. Some or all of these functional blocks may be implemented with various numbers of hardware and/or software configurations that perform particular functions. For example, the functional blocks of the present invention may be implemented by one or more microprocessors, or by circuit configurations for a predetermined function. The functional blocks of the present disclosure may be implemented in various programming or scripting languages. The functional blocks of the present invention may be implemented with algorithms running on one or more processors. The functions performed by the functional blocks of the present invention may be performed by a plurality of functional blocks, or the functions performed by the plurality of functional blocks in the present invention may be performed by one functional block. In addition, the present invention may employ conventional techniques for electronic configuration, signal processing, and/or data processing, and the like.

The connection lines or connecting members between the components shown in the figures are merely illustrative of functional connections and/or physical or circuit connections. In actual devices, connections between components can be implemented by various functional connections, physical connections, or circuit connections that can be replaced or added.

The present invention proposes a device and a method for inducing sleep by using an electroencephalogram tuning phenomenon.

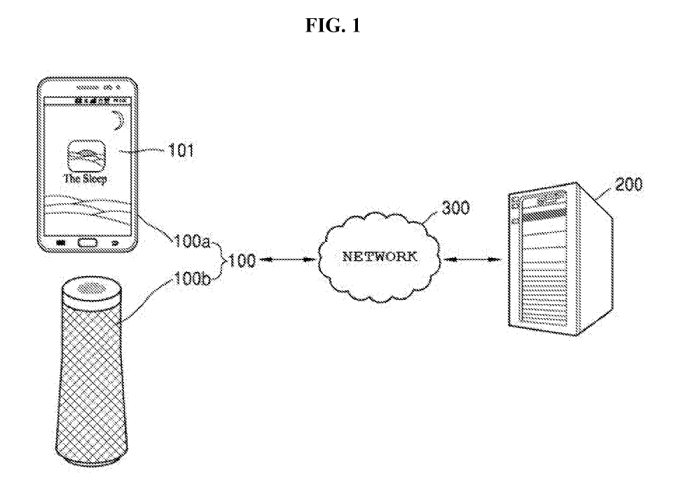

FIG. 1 is a diagram showing a network configuration of a sleep inducing device according to an embodiment.

Referring to FIG. 1, the sleep inducing device 100 according to the present embodiment may output a sleep inducing sound for inducing sleep along with background music. The sleep inducing sound has the maximum amplitude at the inducing frequency of the sleeping, and the pitch can be changed to make a consonance with the melody of the background music.

According to an example, the sleep inducing device 100 can directly output background music and a sleep inducing sound in a sound form using an audio output device (e.g., a speaker) installed therein.

According to another example, when the sleep inducing device 100 is connected to an external audio device (e.g., an external speaker device, a Bluetooth speaker device, an earphone) including an audio output device, And a second audio signal corresponding to the sleep inducing sound may be output to the external audio device in a wireless or wired manner.

The sleep inducing device 100 can output a mixed audio signal, which is a mixture of the first audio signal and the second audio signal, to an external device in a wireless or wired manner.

In the following, the example in which the sleep inducing device 100 directly outputs the background music and the sleep inducing sound in a sound form is mainly described. However, the inventive idea of the present invention is not limited thereto, and the sleep inducing device 100 may transmit the audio signal corresponding to the background music and the sleep inducing sound to the outside in a wired or wireless manner.

The sleep inducing sound may consist of sound pulses generated corresponding to the sleep inducing frequency. For example, if the sleep inducing frequency is assumed to be 3 Hz, the sleep inducing sound may include three sound pulses consecutively generated for one second.

Each sound pulse may include a sound wave having a predetermined pitch. At the start and end points of one sound pulse, the sound wave has the minimum amplitude, and in the middle of one sound pulse, the sound wave can have the maximum amplitude. For example, the amplitude of a sound wave in each sound pulse may be changed to a sinusoidal shape, where the sound wave may have maximum amplitude at the center of each sound pulse. According to another example, the amplitude of a sound wave in each sound pulse may change in the form of at least one of a triangle wave, a sawtooth wave, and a square wave.

Each of the sound pulses may include sound waves of a predetermined pitch corresponding to the melody of the background music output together with the sleep inducing sound. For example, the sleep inducing sound may include sound pulses composed of sound waves of a first pitch during a first time interval, and sound pulses of sound waves of a second pitch during a second time period after the first time interval. Therefore, the pitch of the sleep inducing sound can be changed so as to make a consonance the melody of the background music.

The sleep inducing device 100 may be, for example, a wireless communication terminal 100a such as a smart phone.

The wireless communication terminal 100a is provided with an application program (hereinafter referred to as a ‘sleep inducing application’) that receives user input and provides user interface (UI) for outputting background music and a corresponding sleep inducing sound according to user input can be installed. The wireless communication terminal 100a may access the server 200 providing an app store or a play store through the communication network 300 to download the sleep inducing application.

According to another example, the wireless communication terminal 100a may download the sleep inducing application via a separate storage medium, or may download the sleep inducing application through wired/wireless communication with another device.

The wireless communication terminal 100a in which the sleep inducing application is installed can execute the sleep inducing application when the preset user input is received. When the sleep inducing application is executed, the wireless communication terminal 100a may display a user interface on the display unit 101 so that the user can select desired background music. When the user selects desired background music through the user interface, the wireless communication terminal 100a can output the sleep inducing sound to the speaker together with the selected background music. When the earphone or the speaker device is connected to the audio jack of the wireless communication terminal 100a or the wireless speaker device is connected using the Bluetooth, the wireless communication terminal 100a transmits the background music along with the sleep inducing sound can be output in a soundforms.

The sleep inducing sound has sound pulses corresponding to the sleep inducing frequency, and each of the sound pulses can be composed of a sound wave having a pitch which makes to a consonance with a melody of the background music which is outputted with the sleep inducing sound.

Accordingly, the pitch of sleep inducing sound can be changed according to the melody of background music.

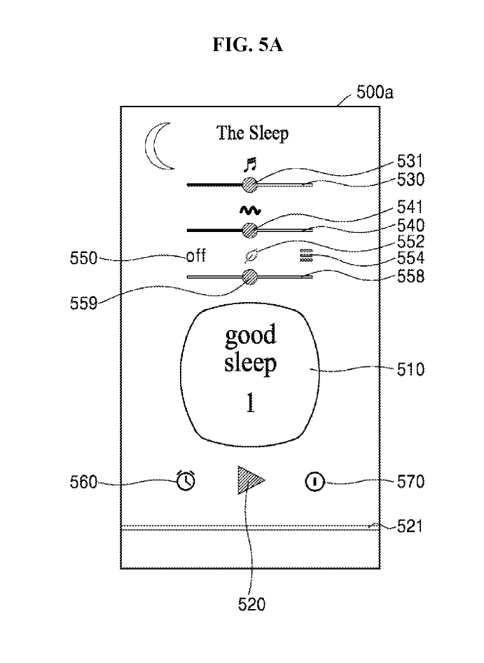

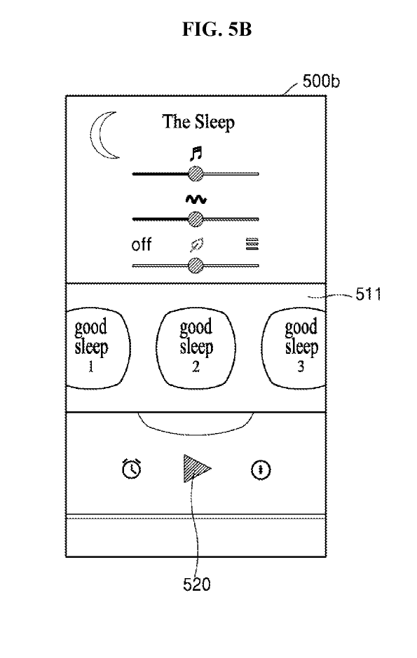

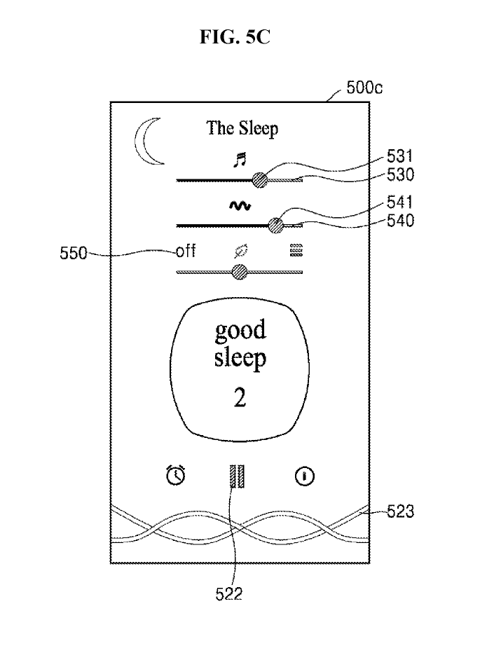

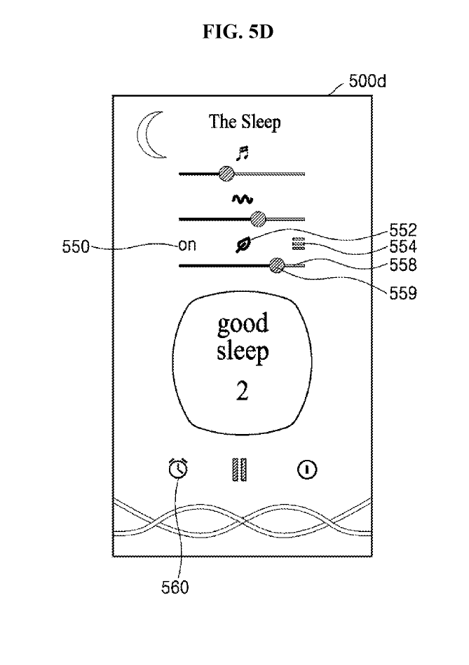

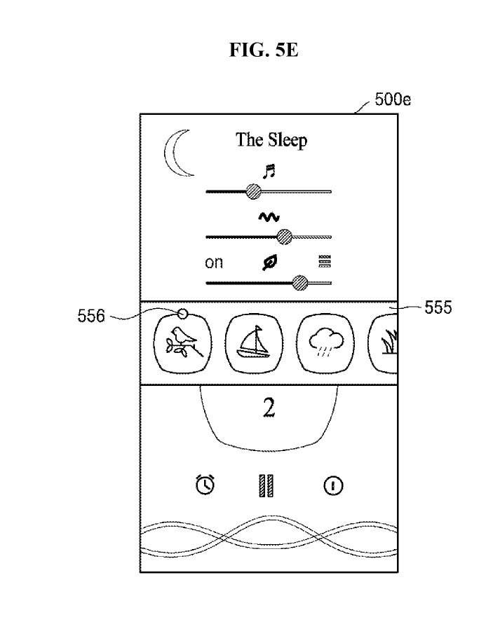

The user interface provided by the wireless communication the terminal 100a functioning as the sleeping guide device 100 will be described in more detail below with reference to FIGS. 5 to 7.

The communication network 300 may include a wired network and/or a wireless network. For example, the communication network 300 may include various wired/wireless networks such as a personal area network (PAN), a local area network (LAN), a metropolitan area network (MAN) and a wide area network (WAN), and so on.

The communication network 300 may be connected to the World Wide Web. However, the communication network 300 according to the present embodiment is not limited to the networks listed above, and may include at least one of a known wireless data network, a known telephone network, and a known wired/wireless television network.

The sleep inducing device 100 may be, for example, a speaker device 100b such as a wireless speaker or a smart speaker. The speaker device 100b may be provided with a program (hereinafter referred to as a “sleep inducing program”) for receiving a user input and outputting a background music and a sleep inducing sound corresponding to the background music according to the user input. The speaker device 100b may have a sleep inducing mode. When the sleep inducing mode is selected, the speaker device 100b can output the sleep inducing sound in a sound form together with the background music according to the sleep inducing program.

The sleep inducing program may be downloaded from the server 200 via the communication network 300 and installed in the speaker device 100b or may be installed in the speaker device 100b through wired or wireless communication with other computing devices. The speaker device 100b may have an audio playback mode. When the audio playback mode is selected, an audio file stored in the speaker device 100b may be played or an audio signal received from another device connected via wire/wireless communication may be played. According to another example, the sleep inducing device 100 may be an independent dedicated device for inducing sleep of the user.

The sleep inducing device 100 described herein includes a mobile phone, a smart phone, a smart speaker, a smart TV, a laptop computer, a personal digital assistant (PDA), a portable multimedia player (PMP).

Although FIG. 1 shows a configuration in which the sleep inducing device 100 is connected to the server 200 via the communication network 300, the sleep inducing device 100 can output background music and a sleep inducing sound according to a user input even in an off-line state not connected to the communication network 300.

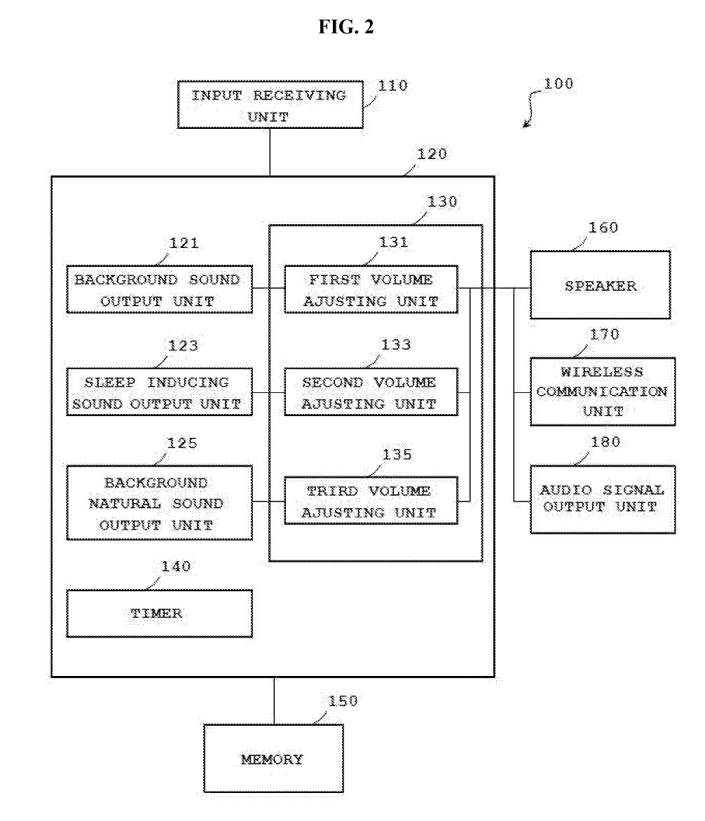

FIG. 2 is a block diagram showing a configuration of a sleep inducing device according to an embodiment.

Referring to FIG. 2, the sleep inducing device 100 includes an input receiving unit 110, a control unit 120, and a memory 150. The control unit 120 includes a background sound output unit 121, a sleep inducing sound output unit 123, and a mixing unit 130. The sleep inducing device 100 may further include components other than the components 110-180 shown in FIG. 2. The control unit 120 may be implemented as a processor that executes the commands stored in the memory 150.

The input receiving unit 110 may receive a user input. If the sleep inducing device 100 is a wireless communication terminal 100a such as a smart phone, the user input may be received through the touch screen of the display unit 101. According to another example, the user input may be received via the voice recognition function of the wireless communication terminal. If the sleep inducing device 100 is a speaker device 100b or an independent dedicated device, the user input may be received via the input button and may be received in the form of a voice command if the device has a voice recognition function. The user input may include an input for executing a sleep inducing device or a program, an input for selecting a background music, an input for independently controlling the volume of background music and a sleep inducing sound, a background input for selecting a natural sound, an input for setting the menu, an input for setting the timer, a sleep inducing application, or an input for ending the program, etc.

The input receiving unit 110 may include a user input unit (e.g., a touch key, a mechanical key, and the like) for receiving information from a terminal user.

The input receiving unit 110 may include a camera or an image input unit for inputting a video signal, a microphone or an audio input unit for inputting an audio signal, and the like. The data received through the input receiving unit 110 is analyzed by the control unit 350 and can be processed by a control command of the user of terminal.

The control unit 120 may control the overall operation of the sleep inducing device 100. The control unit 120 can control an operation related to an application program or an application stored in the memory 150. The control unit 120 performs functions of the background sound output unit 121, the sleep inducing sound output unit 123 and the mixing unit 130 to implement the sleep inducing device 100 according to various embodiments of the present invention. The control unit 120 may control components such as the input receiving unit 110, the memory 150, the speaker 160, and the like.

The control unit 120 may drive the sleep inducing application stored in the memory 150. The control unit 120 can generate information (e.g., screen configuration information) about the user interface (UI) according to the sleep inducing application. The user interface UI may include an area for allowing the user to select desired background music, an area for allowing the user to stop or resume playback of the background music and the sleep inducing sound, an area for allowing the setting to be changed, and the like. The screen configuration information may include information on the structure and arrangement of graphic objects constituting the user interface (UI), items included in the user interface, information on the contents, and the like. The screen configuration information can be produced by UI commands. The command may be a web script produced in various web languages such as Hypertext Markup Language (HTML), Cascading Style Sheets (CSS), JavaScript, XML (eXtensible Markup Language).

The memory 150 may store data supporting various functions of the sleep inducing device 100. The memory 150 may store an application program or an application installed in the sleep inducing device 100, data and commands for operation of the sleep inducing device 100, and the like. According to the present embodiment, the memory 150 may store a sleep inducing application or the like. Further, the memory 150 may store a plurality of background music data, and a plurality of sleep inducing sound data respectively corresponding to the plurality of background music data.

The memory 150 can include at least one media of a flash memory type, a hard disk type, a solid state disk type, an SDD type (Silicon Disk Drive type), a multimedia card micro type, card type memory (e.g., SD or XD memory), random access memory (RAM), static random access memory (SRAM), read-only memory (ROM), electrically erasable programmable read-only memory (EEPROM), a programmable read-only memory (PROM), a magnetic memory, a magnetic disk, and an optical disk.

The background sound output unit 121 may output the first signal corresponding to the background music selected according to the user input. The background sound output unit 121 may read the background music data corresponding to the background music selected according to the user input from the memory 150. The background sound output unit 121 may generate a first signal corresponding to the read background music data. The first signal may be referred to as a first audio signal. The background music selected by the user input is referred to as the first background music.

The background music is music with melody and rhythm, and it can be music that can help induce sleep. For example, background music can be a monotonous and slow tempo music, such as a lullaby or a prenatal music.

The background music may include autonomous sensory meridian response (ASMR). It is known that the autonomous sensory meridian response (ASMR) induces psychological stabilization by stimulating the brain. The autonomous sensory meridian response (ASMR) may include, for example, a beating sound, a writing with a pencil, a rustling sound, a door opening sound, or a whispering sound. The autonomous sensory meridian response (ASMR) may include sounds referred to as a relaxation sound or an ambience sound.

The sleep inducing sound output unit 123 may output a second signal corresponding to the sleep inducing sound corresponding to the first background music. The sleep inducing sound output unit 123 can read the sleep inducing sound data corresponding to the first background music from the memory 150. The sleep inducing sound output unit 123 may generate a second signal corresponding to the read sleep inducing sound data. The second signal may be referred to as a second audio signal. The sleep inducing sound corresponding to the first background music is referred to as a first sleep inducing sound. The first signal and the second signal may be electrical signals or may be in the form of digital data.

The sleep inducing sound can have maximum amplitude at the sleep inducing frequency. The pitch of the sleep inducing sound can be changed so as to make a consonance the melody of the background music. The consonance means a harmonious sound, and when two or more sounds are sounded together, they are harmonious. The consonance may be described by the negative of dissonance. As the proportion of an oscillation frequency of the two sounds becomes a simple integer, it can be said to be a consonance. For example, it can be regarded as a consonance when it has interval of a perfect 1^{st}, a perfect 5^{th}, a perfect 4^{th}, a major 3^{rd}, a minor 3^{rd}, a major 6^{th}, and a minor 6^{th}.

The sleep inducing sound may have a pitch that varies depending on the output of the corresponding background music. The background music and the sleep induced sound will be described in more detail below with reference to FIG.

The mixing unit 130 may mix the first signal corresponding to the background music and the second signal corresponding to the sleep inducing sound to output a mixing signal. When the first signal and the second signal are electrical signals, the mixing unit 130 may generate a mixing signal by mixing the first signal and the second signal using a summing amplifier circuit. When the first signal and the second signal are digital data, the mixing unit 130 performs an audio mixing function on the first signal and the second signal to generate mixing data.

The mixing unit 130 may output the generated mixing signal to the speaker 160. According to another example, the mixing unit 130 may output the generated mixing signal to the wireless communication unit 170. According to another example, the mixing unit 130 may output the generated mixing signal to the audio signal output unit 180, for example, an audio jack.

The mixing unit 130 may include a first volume control unit 131 and a second volume control unit 133. The first volume control unit 131 may adjust the size of the first signal according to a first volume input of the user. The first volume input of the user is an input for adjusting the volume of the background music. The second volume control unit 133 may adjust the size of the second signal according to the second volume input of the user. The second volume input of the user is an input for adjusting the volume of the sleep inducing sound. The volume of the background music and the volume of the sleep inducing sound can be independently controlled by the first volume control unit 131 and the second volume control unit 133. The mixing unit 130 may generate a mixing signal by mixing a first signal whose magnitude is adjusted by the first volume control unit 131 and a second signal whose magnitude is adjusted by the second volume control unit 133. Therefore, the user can freely adjust the volume of the background music and the volume of the sleep inducing sound according to his or her tendency or preference.

The sleep inducing device 100 may include at least one of a speaker 160, a wireless communication unit 170, and an audio signal output unit 180. The speaker 160 receives the mixing signal generated by the mixing unit 130, and can directly output audio mixed with the background music and the sleep inducing sound in a sound form. The wireless communication unit 170 can communicate with the wireless audio device. The wireless communication unit 170 receives the mixing signal generated by the mixing unit 130, converts the mixed signal into a wireless signal, and transmits the wireless signal to the wireless audio device. For example, a speaker device or an earphone may be connected to the audio signal output unit 180, and the audio signal output unit 180 may output the electric mixing signal generated by the mixing unit 130 to a speaker device or an earphone. The audio signal output unit 180 may be, for example, an audio jack to which an audio plug terminal can be connected.

The control unit 120 may further include a background natural sound output unit 125. The background natural sound output unit 125 may output a third signal corresponding to the background natural sound according to a user input. The memory 150 may store a plurality of background natural sound data. The background natural sound output unit 125 may read the background natural sound data selected in accordance with the user input from the memory 150 to generate the third signal. Background Natural sounds may include winds, birds singing, rains, insects in the woods, and firewood. In addition, background natural sounds may include waterfall sounds, train sounds, library sounds, and office sounds.

The mixing unit 130 mixes the first signal output from the background sound output unit 121, the second signal output from the sleep inducing sound output unit 123, and the third signal output from the background natural sound output unit 123, and can generate a mixing signal.

The mixing unit 130 may further include a third volume control unit 135. The third volume control unit 135 may adjust the size of the third signal according to the third volume input of the user. The third volume input of user is an input for adjusting the volume of the background natural sound. Thus, the volume of the background music, the volume of the sleep inducing sound, and the volume of the background natural sound can be adjusted independently of each other.

The control unit 120 may further include a timer 140. The user can use the timer 140 to automatically set the time during which the sleep inducing application or program ends. Since the sleep inducing sound is for inducing sleep, it can be helpful not to continuously listen to sleep inducing sound and background music while the user is sleeping. Therefore, the user can automatically terminate the sleep inducing application or the program using the timer (140).

Although not shown in FIG. 2, the sleep inducing device 100 may further include a display unit (101 in FIG. 1). The display unit 101 outputs or displays information processed by the sleep inducing device 100. For example, the display unit 101 may display execution screen information, user interface (UI) information, and GUI (Graphic User Interface) information of an application program driven by the sleep inducing device 100.

The display unit 101 may have a laminated structure with the touch panel or may be formed as a touch screen by being integrated with the touch panel. The touch screen may provide an input interface and output interface between the sleep inducing device 100 and the user.

The wireless communication unit 170 of the sleep surface inducing device 100 can perform a function of connecting various communication networks or other devices in addition to transmitting the mixing signal wirelessly. The wireless communication unit 170 may include a mobile communication module, a short range communication module, and a wireless Internet module. The sleep inducing device 100 can download the sleep inducing application installation program from the server 200 via the wireless communication unit 170.

The mobile communication module can transmit and receive a radio signal to and from the adjacent base stations in a mobile communication network established according to a technical standard or a communication method for mobile communication (for example, Global System for Mobile communication (GSM), Code Division Multi Access (CDMA), Code Division Multi Access 2000 (CDMA2000), Enhanced Voice-Data Optimized or Enhanced Voice-Data Only (EV-DO), Wideband CDMA (WCDMA), High Speed Packet Access (HSPA), Long Term Evolution (LTE), Long Term Evolution-Advanced (LTE-A).

The short range communication module is for short range communication, and can support short range communication using at least one of Bluetooth™, Radio Frequency Identification (RFID), Infrared Data Association (IrDA), Ultra Wideband (UWB), ZigBee, Near Field Communication (NFC), Wireless-Fidelity (Wi-Fi), Wi-Fi Direct, and Wireless Universal Serial Bus (Wireless USB) technologies. For example, the wireless communication unit 170 may transmit the mixing signal to the wireless speaker device through the Bluetooth module.

The wireless Internet module is a module for wireless Internet access, and is configured to transmit and receive wireless signals in a communication network according to wireless Internet technologies. Wireless Internet technologies include, for example, Wireless LAN (WLAN), (Wireless-Fidelity (Wi-Fi), Wi-Fi Direct, Digital Living Network Alliance (DLNA), Wireless Broadband (WiBro) World Interoperability for Microwave Access (WiMAX), and the like. And the wireless Internet module can transmit and receive data according to at least one wireless Internet technology, including Internet technologies not described above.

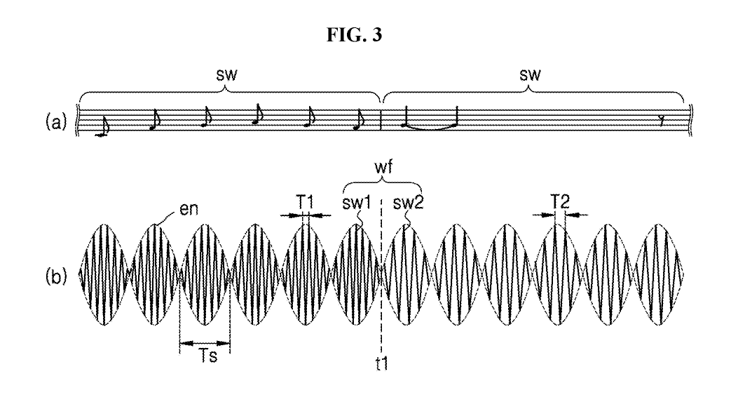

FIG. 3 is a diagram for explaining background music and a sleep inducing sound output by the sleep inducing device according to an embodiment.

In (a) of FIG. 3, a part of the sheet music is illustratively shown. The background music may have a melody and rhythm according to the sheet music shown in (a) of FIG. 3. In FIG. 3, (a) does not limit the present invention as an example of sheet music. Although (a) of FIG. 3 illustrates that there is no chord, background music may have chords other than melody and rhythm.

In (b) of FIG. 3, the waveform (wf) of the sleep inducing sound is exemplarily shown. The waveform (wf) of the sleep inducing sound is represented by a solid line and the envelope (en) of the sleep inducing sound is indicated by a dashed line.

The envelope (en) represents the change in the amplitude of the sound waves sw1 and sw2, that is, the change in the intensity of the sound inducing sound. When the width of the envelope (en) is small, it indicates that the strength of the sleep inducing sound is small and the strength of the sleep inducing sound is large when the width of the envelope (en) is large. The envelope (en) has a period (Ts) corresponding to the sleep inducing frequency and the sound waves sw1 and sw2 of one period (Ts) can be referred to as sound pulses. The user who listens to the sleep inducing sound output by the sleep inducing device 100 can hear the sound having the pitch corresponding to the frequency of the sound waves sw1 and sw2 every period (Ts). As shown in (b) of FIG. 3, the envelope (en) may have a sinusoidal form. The envelope (en) can have a magnitude corresponding to the absolute value of the sine wave. However, if it is not limited to this, the envelope (en) may have a shape such as a triangle wave, a sawtooth wave, a square wave, or the like.

The waveform (wf) of the sleep inducing sound shown in (b) of FIG. 3 can be formed by periodically increasing and decreasing the intensity of the sound having the pitch set to make a consonance with the melody periodically (Ts). The sleep inducing sound can be produced using a synthesizer or the like.

The first sound wave (sw1) of the sleeping inducing sound has a first frequency having the first pitch during the first section (TA) before the first time (t1), and the second sound wave (sw2) of the sleep inducing sound may have a second frequency having the second pitch during the second section (TB) after the first time (t1).

As shown in (b) of FIG. 3, the first sound wave (sw1) has a first period (T1) corresponding to the first frequency and the second sound wave (sw2) has a second period (T2) corresponding to the second frequency. As shown in (b) of FIG. 3, when the second period (T2) is longer than the first period (T1), the second frequency of the second sound wave (sw2) is lower than the first frequency of the first sound wave (sw1). In this case, the first pitch of the first sound wave (sw1) is lower than the second pitch of the second sound wave (sw2).

The first pitch of the first sound wave (sw1) and the second pitch of the second sound wave (sw2) may be determined according to the melody of the background music. The first and second pitches can be set in advance so as to have a consonance with melody of the background music to be played back together. In the example shown in FIG. 3, the melody of the background music changes to Do, Fa, Sol, La, Sol, Fa during the first section (TA), and the melody of the background music during the second section (TB) is Sol. During the first section (TA), the first pitch of the first sound wave (sw1) can correspond to “Fa” so as to be in consonance with melody of the background music of the first section (TA), and during the first section (TA), the second pitch of the second sound wave (sw2) can correspond to “Do” so as to be in consonance with melody of the background music of the second section (TB).

Thus, the pitch of the sleep inducing sound changes in consonance with the melody of the background music, so that the user can feel comfortable. However, unlike the present invention, when the pitch of the inducing sleep sound is constant, that is, in the second section (TB), if the sleep inducing sound has a pitch corresponding to ‘Fa’ as in the first section (TA), the background music having the pitch of the ‘Sol’ and the sleep inducing sound having the pitch of ‘Fa’ are outputted together. Since the ‘Sol’ and the ‘Fa’ make a dissonance of the major 2^{nd}, the user will hear an unpleasant sound during the second section (TB). Thus, the dissonance generated by the background music and the sleep inducing sound may cause the user to wake up from the sleep.

The sleep inducing frequency may be selected within a preset frequency range to induce sleep. For example, the sleep inducing frequency may be selected between 2 Hz and 10 Hz. The sleep inducing frequency may be selected between 2.5 Hz and 4 Hz.

When the user hears the sleep inducing sound whose amplitude changes with a certain inducing frequency, the frequency of the EEG is changed to be the same as the sleep inducing frequency by the EEG tuning phenomenon.

It is known that a human EEG exhibits a seta wave between 4 Hz and 8 Hz in a drowsy state or a shallow sleep state, and exhibits a delta wave between 0.5 Hz and 4 Hz in an unconscious state in a deep sleep. By outputting a sleep inducing sound at a frequency between 2 Hz and 10 Hz or between 2.5 Hz and 4 Hz, the user can be brought to a drowsy state immediately before falling asleep, to a shallow sleeping state, or to a deep sleeping state.

The sleep inducing frequency may be set to decrease after a predetermined time. For example, during the first 10 minutes, the sleep inducing device 100 outputs a sleep inducing sound with a sleep inducing frequency of 6 Hz, outputs a sleep inducing sound with a sleep inducing frequency of 4 Hz during the next 10 minutes, and outputs a sleep inducing sound at a sleep inducing frequency of 2.5 Hz during the next 10 minutes. As the sleep inducing frequency decreases, the tempo of the background music may also be slowed down. However, the speed of the background music may be fixed.

According to another example, the sleep inducing frequency may be set to decrease by 10% after the background music is repeated a predetermined number of times. For example, while the background music is repeated three times, the sleep inducing frequency is set to 8 Hz, while the background music is repeated next three times, the sleep inducing frequency is set to 7.2 Hz, and while the background music is repeated next three times, the sleep inducing frequency is set to 6.4 Hz. In this case, the speed of the background music may also be slowed down or kept constant.

According to another example, the sleep inducing frequency may gradually become slower with time after a predetermined time. In this case as well, the background music may be slowed together in correspondence to the sleep inducing frequency.

When the sleep inducing frequency is, for example, 4 Hz, one period (Ts) corresponds to 250 ms. If the pitch of the inducing sound changes in the middle, the user can detect the moment when the pitch changes, and the moment when the pitch changes can give the user an uncomfortable feeling or unnatural feeling. According to an example, the pitch of the sleep inducing sound may be set to change between the period (Ts) and the period (Ts). That is, the sound pulses constituting the sleep inducing sound may have the same pitch.

According to another example, the pitch of the sleep inducing sound may change when the envelope (en) is less than a preset ratio relative to the maximum value. For example, if the maximum value of the envelope (en) is 1, when the envelope (en) is 0.2 or less, the pitch of the sleep inducing sound may change. The envelope (en) corresponds to the amplitude of the sound wave of the sleep inducing sound, and thus represents the intensity of the sleep inducing sound. When the intensity of the sleep inducing sound is not large, the pitch changes, and the user is difficult to notice that the pitch changes.

The pitch of the sleep inducing sound changes according to the melody of the background music. The background music may be divided into nodes corresponding to a plurality of beats, as shown in (a) of FIG. 3. The pitch of the sleep inducing sound is likely to change when a new node is started. Although it is not the moment when a new node begins, the pitch of the sleep inducing sound is likely to change when a new beat begins. The frequency per minute corresponding to the sleep inducing frequency may be an integral multiple of the number of beats per minute of the background music so that the pitch can be changed at the moment of start of a new sound pulse. For example, if the sleep inducing frequency is 3 Hz, the frequency per minute corresponds to 180. In this case, the number of beats per minute of background music may be 180, 90, 60, 45, and so on. In the example of FIG. 3, the number of beats per minute of the background music is 90. Thus, during one beat of the background music, two sound pulses can be output from the sleep inducing sound.

According to an example, the OSC level may be set at about 29%, and the portamento time at about 160 ms, to make naturally the moment at which the pitch of the sleep inducing sound changes. Also, with respect to the envelope (en), the attack time may be set to about 151 ms, the decay time may be set to about 1 s, and the release time may be set to about 1.11 s. The sustain can be set to about 0 db.

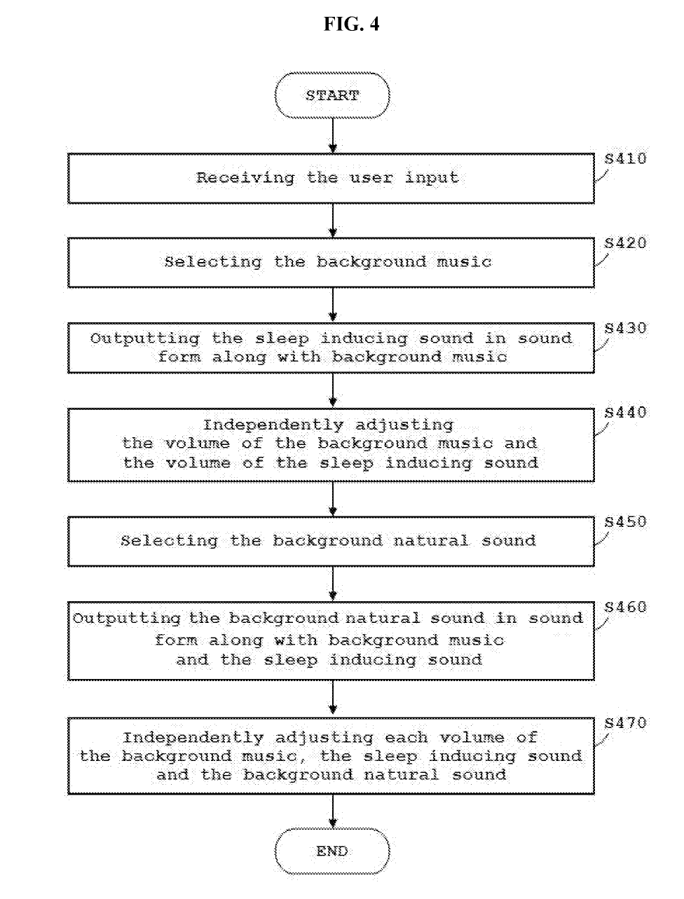

FIG. 4 is a flowchart illustrating a sleep inducing method according to an embodiment.

Referring to FIG. 4 together with FIG. 2, the input receiving unit 110 may receive a user input (S410). The control unit 120 can confirm the user input received by the input receiving unit and perform an operation corresponding to the user input. For example, the user input may be a request to start a sleep inducing program, and the control unit 120 may drive the sleep inducing program in response to such user input.

Also, the user input may be an input for selecting background music (S420). The control unit 120 may read the background music data corresponding to the selected background music data stored in the memory 150. In addition, the control unit 120 may read the sleep inducing sound data corresponding to the selected background music from the sleep inducing sound data stored in the memory 150. The control unit 120 may generate the first audio signal and the second audio signal based on the read background music data and the read sleep inducing sound data. The first audio signal may be an audio signal corresponding to the read background music data and the second audio signal may be an audio signal corresponding to the read sleep inducing sound data.

The control unit 120 mixes the first audio signal and the second audio signal to generate a mixed audio signal, and may output the mixed audio signal in a sound form using the speaker 160 (S430). The control unit 120 may output the mixed audio signal as a wireless signal or a wired electric signal to the other speaker device using the wireless communication unit 170 or the audio signal output unit 180. The sleep inducing sound corresponding to the second audio signal may be changed in pitch so as to make a consonance with the melody of the background music corresponding to the first audio signal. In addition, the sleep inducing sound may include sound pulses that occur in accordance with the sleep inducing frequency.

The control unit 120 may receive a user input for adjusting the volume of the background music and/or the volume of the sleep inducing sound through the input receiving unit 110. When the user input is received, the control unit 120 can independently adjust the amplitude of the first audio signal and/or the amplitude of the second audio signal (S440). The control unit 120 may adjust the amplitude of the first audio signal to adjust the volume of the background music and the amplitude of the second audio signal to adjust the volume of the sleep inducing sound. The volume of the background music and the volume of the sleep inducing sound can be adjusted independently of each other. The sleep inducing sound may be adjusted to be silent so that only the background music is output, or the background music is adjusted to be silent so that only the sleep inducing sound is output.

The control unit 120 may receive a user input for selecting a background natural sound through the input receiving unit 110 (S450). In response to the user input, the control unit 120 may read the background natural sound data corresponding to the background natural sound selected from the background natural sound data stored in the memory 150. The control unit 120 may generate the third audio signal based on the read background natural sound data.

The control unit 120 may add a third audio signal to the mixed audio signal. That is, the control unit 120 may mix the first through third audio signals to generate a new mixed audio signal. The control unit 120 may output the generated mixed audio signal in a sound form through the speaker 160 (S460).

The control unit 120 may receive user input for independently controlling the volume of each of the background music, the sleep inducing sound, and the background natural sound through the input receiving unit 110. The control unit 120 may adjust the amplitude of each of the first to third audio signals corresponding to the user input. Accordingly, the user can independently adjust the volume of each of the background music, the sleep inducing sound, and the background natural sound (S470).

The sleep inducing device 100 shown in FIG. 1 can operate according to the sleep inducing method described with reference to FIG. 4.

Hereinafter, referring to FIG. 5 to FIG. 7, operations of the sleep inducing device will be described in detail using a user interface (UI) screen when the sleep inducing device according to the present invention is a wireless communication terminal equipped with a sleep inducing application.

FIGS. 5A to 5E illustrate a user interface screen of a sleep inducing device in which a sleep inducing application is installed according to an embodiment.

The screen 500a shown in FIG. 5A is an initial screen in which the user selects an icon corresponding to the sleep inducing application and drives the sleep inducing application.

The background music region 510 may display the currently selected background music. The first background music may be selected by default and displayed in the background music region 510 as shown in FIG. 5A. According to another example, recently selected background music may be displayed in the background music area 510. When the background music region 510 is selected, the user can select background music as shown in the selection window 511 of FIG. 5B. The user can select the selectable background music listed on the left and right in the selection window 511. By moving the selection window 511 to the left and right, the user can display more selectable background music on the screen.

The play icon 520 is an icon for playing the background music displayed in the background music area 510. When the user selects the play icon 520, the background music displayed in the background music area 510 and the corresponding sleep inducing sound can be played back. The screen 500a shown in FIG. 5A is a screen before the background music is played back, and the area 521 indicates that no sound is played back. For reference, when the sleep inducing sound is being played back, the waveform can be displayed as in the area 523 of FIG. 5C.

The background music volume adjustment area 530 is an area for the user to adjust the volume of the background music. The user can independently adjust the volume of the background music by moving the mark 531 displayed in the background music volume adjustment area 530 to the left and right.

The sleep inducing sound volume adjusting area 540 is an area for the user to adjust the volume of the sleep inducing sound. The user can independently adjust the volume of the sleep inducing sound by moving the mark 541 displayed in the sleep inducing sound volume control area 540 to the left and right.

The background natural sound activation area 550 is an area for determining whether or not the user adds a background natural sound. As shown in FIG. 5A, when the background natural sound activation area 550 is displayed as OFF, the background natural sound may be inactivated. When the user selects the background natural sound activation area 550, as shown in FIG. 5D, the background natural sound activation area 550 is displayed as ON and the background natural sound can be played back. In addition, as shown in FIG. 5D, the background natural sound icon 552 may be brightly displayed to indicate that the background natural sound is active. In FIG. 5A, the background natural sound icon 552 is darkened to indicate that the background natural sound is in an inactive state. In the state where the background music and the sleep inducing sound are being played back, when the user activates the background natural sound by selecting the background natural sound active area 550 indicated as OFF, the background natural sound may be played back together with the background music and the sleep inducing sound.

The background natural sound selection icon 554 is an icon for selecting a background natural sound. When the user selects the background natural sound selection icon 554, a selection window 555 in which selectable background natural sounds are listed as shown in FIG. 5E may be displayed. The mark 556 may represent the currently selected background natural sound. Selectable Backgrounds Natural sounds may include new sounds, waves, rain, forest sounds, bonfires, clocks, caverns, and vinyl sounds.

The background natural sound volume control area 558 is activated when the background natural sound is activated, and is an area for the user to adjust the volume of the background natural sound. The user can adjust the volume of the background natural sound independently by moving the mark 559 displayed in the background natural sound volume control area 558 to the left and right.

The timer icon 560 is an icon for setting the time for automatic termination. If the help icon 570 is selected, a description of the sleep inducing application may be displayed.

The screen 500b of FIG. 5B is displayed when the user selects the background music area 510 on the screen 500a of FIG. 5A. As shown in FIG. 5B, a selection window 511 is displayed, and the user can select a desired background music through the selection window 511.

FIG. 5C is a screen displayed when the user selects the second background sound and selects the play icon 520 in the selection window 511 displayed on the screen 500b of FIG. 5B. The stop icon 522 is displayed at the portion where the play icon 520 is displayed in the screen 500b of FIG. 5B. When the user selects the stop icon 522, the background music and the sleeping inducing sound which has been played back can be stopped. In addition, since the second background sound is being played back, a waveform may be displayed in the area 523 in order to display it.

The user can independently adjust the volume of the background music and the volume of the sleep inducing sound by using the mark 531 of the background music volume control area 530 and the mark 541 of the sleep inducing sound volume control area 540. In FIG. 5C, the state in which the volume of the sleep induction sound is adjusted to about 80% and the volume of the background music is adjusted to about 60% is displayed.

The screen 500d shown in FIG. 5D is displayed when the user selects the background natural sound activation area 550 on the screen 500c in FIG. 5C. As shown on the screen (500d) of FIG. 5D, the background natural sound is activation area (550) is displayed ON, and the background natural sound icon (552) can be displayed bright. The user can independently adjust the volume of the background natural sound regardless of the volume of the background music and the volume of the sleep inducing sound by using the mark 559 of the background natural sound volume control area 558. In FIG. 5D, the state in which the volume of the background music is adjusted to about 40%, the volume of the sleep inducing sound is adjusted to about 60%, and the volume of the background natural sound is adjusted to about 80% is displayed.

The screen 500e of FIG. 5E is displayed when the user selects the background natural sound selection icon 554 on the screen 500d of FIG. 5D. The selection window 555 is displayed as shown on the screen 500e of FIG. 5E, and the user can select any of the background natural sounds displayed in the selection window 555. The mark 556 indicates the currently selected background natural sound, and in FIG. 5E, a bird sound is selected as the background natural sound.

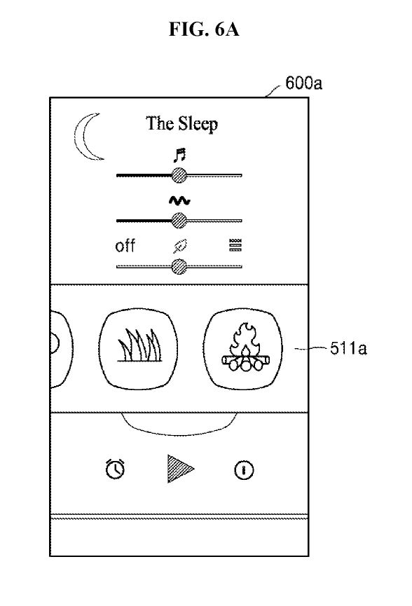

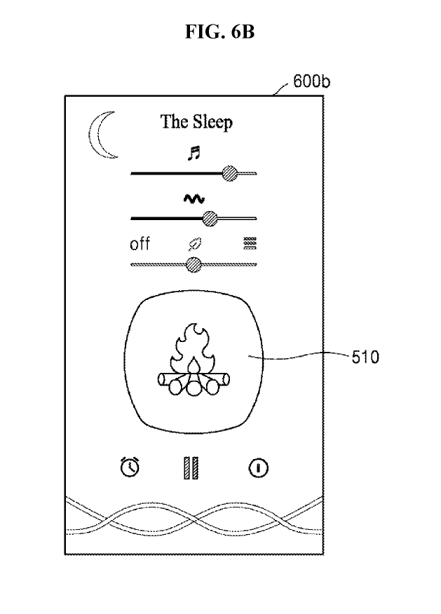

FIGS. 6A-6B illustrate a user interface screen of a sleep inducing device in which a sleep inducing application is installed according to an embodiment.

FIG. 6A is a screen displayed when the user selects the background music area 510 on the screen 500a of FIG. 5A. As shown in FIG. 6B, a selection window 511a is displayed. When the user adjusts the selection window 511a to the left or right, selectable items can be displayed on the screen 600a. As with the items displayed in the selection window 511a, the user may select only background natural sounds without background music.

The screen 600b of FIG. 6B is displayed when the user selects bonfire sound as background music in the selection window 511a displayed on the screen 600a of FIG. 6A. As shown in the screen 600b of FIG. 6B, an icon symbolizing a bonfire is displayed in the background music area 510, and a sleep inducing sound may be played back together with a bonfire sound. At this time, unlike background music, since the sound of bonfire does not have a melody, the pitch of the sleep inducing sound can be constant without changing.

According to another embodiment, the user may select an audio book to be read by a voice actor in the selection window 511a. In this case, the sleep inducing sound may be played back together with the audio book sound read by the voice actor. At this time, unlike the background music, since the audio book sound does not have a melody, the pitch of the sleep inducing sound can be constant without changing. In addition, audio book sound, sleep inducing sound, as well as background natural sound can be further output.

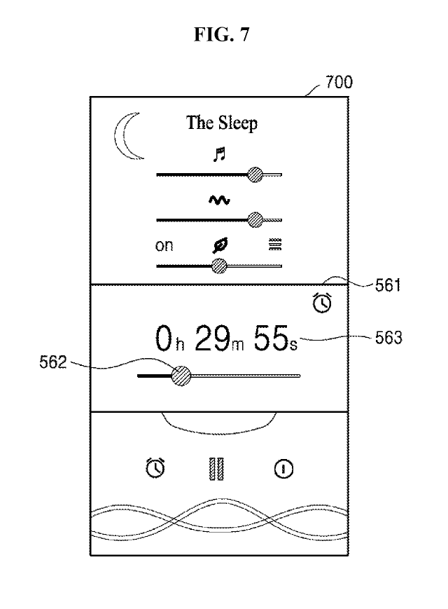

FIG. 7 illustrates a user interface screen of a sleep inducing device in which a sleep inducing application is installed according to an embodiment.

FIG. 7 is a screen displayed when the user selects the timer icon 560 on the screen 500d of FIG. 5D. As shown in FIG. 7, a timer setting window 561 can be displayed. In the timer setting window 561, a mark 562 for setting the end time can be displayed. The user can set the remaining time until the sleep inducing application is terminated by moving the mark 562 to the left or right. In the area 563 of the timer setting window 561, the remaining time until termination can be displayed. As time passes, the time in area (563) can gradually decrease.

The various embodiments described above may be embodied in the form of a computer program that can be executed on a computer through various components, and such a computer program may be recorded on a computer-readable medium. At this time, the medium may be a program that continuously stores a computer executable program, or temporarily stores the program for execution or downloading. In addition, the medium may be a variety of recording means or storage means in the form of a combination of a single hardware or a plurality of hardware, and is not limited to a medium directly connected to a computer system, but may be dispersed on a network. Examples of the medium include a magnetic medium such as a hard disk, a floppy disk and a magnetic tape, an optical recording medium such as CD-ROM and DVD, a magneto-optical medium such as a floptical disk, And program commands including ROM, RAM, flash memory, and the like. As another example of the medium, a storage medium can also be mentioned from a recording medium managed by an application store distributing an application, a site that provides or distributes various other software, a server, and the like.

In this specification, “unit”, “module” or the like, may be a hardware component, such as a processor or circuit, and/or a software component, executed by a hardware configuration, such as a processor. For example, “unit”, “module” and the like can be implements by software constituent elements, object-oriented software constituent elements, class constituent elements and task constituent elements, processes, functions, attributes, procedures, subroutines, segments of program code, drivers, firmware, microcode, circuits, data, databases, data structures, tables, arrays, and variables.

It will be understood by those skilled in the art that the foregoing description of the present invention is for illustrative purposes only and that those of ordinary skill in the art can readily understand that various changes and modifications may be made without departing from the idea or essential characteristics of the present invention.

It is therefore to be understood that the above-described embodiments are illustrative in all aspects and not restrictive. For example, each component described as a single entity may be distributed and implemented, and components described as being distributed may also be implemented in a combined form.

It is to be understood that the scope of the present invention is not limited by the above detailed description but is defined by the claims that follow and all changes or modifications that come within the meaning and range of the following claims and their equivalents should be interpreted as being within the scope of.

What is claimed is:

1. A sleep inducing device comprising:
an input receiving unit for receiving a user input;
a background sound output unit for outputting a first signal corresponding to background music having a melody and a rhythm according to the user input;
a sleep inducing sound output unit for outputting a second signal corresponding to a sleep inducing sound having a sinusoidal wave whose pitch changes so as to have consonance with the melody of the background music; and
a mixing unit for mixing the first signal and the second signal to output a mixing signal.

2. The sleep inducing device of claim 1, further comprising:
a memory for storing a plurality of background music data and a plurality of sleep inducing sound data corresponding to the plurality of background music data,
wherein the background sound output unit reads background music data corresponding to the user input from the memory and generates the first signal, and the sleep inducing sound output unit reads the sleep inducing sound data corresponding to the background music data from the memory and generate the second signal.

3. The sleep inducing device of claim 1, wherein the sleep inducing frequency is selected within a predetermined frequency range to induce the sleep.

4. The sleep inducing device of claim 1, wherein the sleep inducing frequency is selected between 2 Hz and 10 Hz.

5. The sleep inducing device of claim 1, wherein a frequency per minute corresponding to the sleep inducing frequency is an integral multiple of beats per minute of the background music.

6. The sleep inducing device of claim 1, wherein the sleep inducing frequency is reduced with a tempo of the background music after a predetermined time.

7. The sleep inducing device of claim 1, wherein the amplitude of the sleep inducing sound changes in a sinusoidal wave form.

8. The sleep inducing device of claim 1, wherein the pitch of the sleep inducing sound changes when the amplitude of the sleep inducing sound is lower than a preset ratio with respect to the maximum amplitude.

9. The sleep inducing device of claim 1, wherein the mixing unit comprising:
a first volume control unit for adjusting a size of the first signal according to a first volume input of the user for adjusting a volume of the background music; and
a second volume control unit for adjusting a size of the second signal according to a second volume input of the user for adjusting the volume of the sleep inducing sound independently of the volume of the background music.

10. The sleep inducing device of claim 1, wherein the background music includes an autonomous sensory meridian response (ASMR).

11. The sleep inducing device of claim 1, further comprising:
a background natural sound output unit for outputting a third signal corresponding to a background natural sound according to the user input, wherein the mixing unit mixes the first to third signals, and outputs the mixing signal.

12. The sleep inducing device of claim 1, further comprising:
a speaker for receiving the mixing signal from the mixing unit and outputting audio mixed with the background music and the sleep inducing sound.

13. The sleep inducing device of claim 1, further comprising:
a wireless communication unit for receiving the mixing signal from the mixing unit, converting the mixing signal into a wireless signal, and transmitting the wireless signal to the wireless audio device.

14. A sleep inducing method comprising:
receiving an user input;
selecting one of the plurality of background music stored in the memory according to an user input; and
mixing a sinusoidal wave sleep inducing sound corresponding to the background music with the background music and outputting them in a sound form,
wherein the background music has a melody, the sleep inducing sound has a maximum amplitude at a sleep inducing frequency, and a pitch is changed so as to make a consonance with a melody of the background music.

15. The sleep inducing method of claim 14, further comprising:
independently adjusting the volume of the background music and the volume of the sleep inducing sound according to the user input.

16. The sleep inducing method of claim 14, further comprising:
selecting one background natural sound of a plurality of background natural sounds stored in the memory according to the user input; and
mixing the background music, the sleep inducing sound, and the background natural sound and outputting them in a sound form.

17. A sleep inducing device comprising:
a processor; and
a memory for storing commands, a plurality of background music data and a plurality of sleep inducing sound data corresponding to each of the plurality of background music data,
wherein the processor is operative to perform the following steps:
receiving a user input when the commands are executed by the processor;
selecting first background music data from a plurality of background music data stored in the memory according to the user input;
selecting first sleep inducing sound data corresponding to the first background music data from a plurality of sleep inducing sound data stored in the memory;
mixing and outputting a background music having a melody corresponding to the first background music data and a sinusoidal wave sleep inducing sound corresponding to the first inducing sleep sound data, and
wherein the sinusoidal wave sleep inducing sound has a maximum amplitude at a sleep inducing frequency, and the pitch changes so as to make a consonance with the melody of the background music.
